(19) United States
(12) Patent Application Publication
(10) Pub. No.: US 2010/0169991 A1
(43) Pub. Date: Jul. 1, 2010

(54) Biopesticide comprising insect belonging to family coccinellidae

(75) Inventors: Teruyuki Niimi, Nagoya-shi (JP); Toshinobu Yaginuma, Aichi (JP)

Correspondence Address: EDWARDS ANGELL PALMER & DODGE LLP, P.O. BOX 55874, BOSTON, MA, 02205, US

(73) Assignee: National University Corporation Nagoya University, Aichi (JP)

(21) Appl. No.: 12/308,664

(22) PCT Filed: Jun. 13, 2007
(86) PCT No.: PCT/JP2007/061851

§ 371 Date: Dec. 19, 2008

(30) Foreign Application Priority Data
Jun. 20, 2006 (JP) 2006 169590

(51) Int. Cl. A01K 67/027; C12N 15/87; A01N 57/16; A01P 7/04

(52) U.S. Cl. 800/13; 800/21; 514/44 A

(57) ABSTRACT

Disclosed is a biopesticide utilizing an insect belonging to the family Coccinelidae, which has excellent durability of effect, which can control a insect pest effectively, and which has little influence on an ecosystem. An insect belonging to the family Coccinelidae having abnormality in the wing formation can be produced by suppressing the expression of a vestigial gene and/or a scalloped gene in the insect.

### TECHNICAL FIELD

The present invention relates to a biopesticide using a natural enemy insect. More particularly, the present invention relates a biopesticide including an insect belonging to family Coccinellidae and a producing method of an insect belonging to family Coccinellidae used for the biopesticide.

### BACKGROUND ART

For controlling pests that are said to extinguish about one-third of agricultural produce, chemical pesticides have been mainly used. However, the chemical pesticides have various harmful influence and problems, for example, much load to the environment, acquisition of resistance, and the like. In order to overcome them, biological methods for pest control using a natural enemy insect have been attempted. As one example, a biopesticide using a ladybug has been put into practical use and used for insect pest control throughout the world. However, when flying and dispersing insects like an adult ladybug are used, it is difficult to obtain a durable effect. Furthermore, the use of such insects has had a risk of influence on the surrounding ecosystem.

Note here that documents related to biopesticides are listed below although they are not related to biopesticides using a ladybug.

[Patent document 1] Japanese Patent Application Unexamined Publication No. 2003-79271

[Patent document 2] Japanese Patent Application Unexamined Publication No. 2002-47116

[Patent document 3] Japanese Patent Application Unexamined Publication No. 2005-272353

### DISCLOSURE OF THE INVENTION

### Problems to be Solved by the Invention

As mentioned above, a biopesticide using a ladybug has had problems in terms of durability of the effect and influence on the ecosystem.

It is therefore a main object of the present invention to provide a biopesticide using a ladybug, which is excellent in durability and permits effective insect pest control as well as has less influence on the ecosystem, and to provide a producing method of a ladybug used for the biopesticide.

### Means for Solving Problems

From the viewpoint of the above-mentioned problems, the present inventors focused on two genes essential to the wing formation of a ladybug, that is, a vestigial gene that is a master gene of the wing formation and a scalloped gene encoding the cofactor thereof. Then, the present inventors have examined the relation between the suppression of expression of these genes and the wing formation by using an RNA interference method (RNAi method) for specifically inhibiting the expression of target genes. Concretely, the present inventors have used Henosepilachna vigintioctopunctata as an experimental model and injected a double-stranded RNA synthesized based on the sequences of the vestigial gene and the scalloped gene into a larva so as to examine the effect thereof. As a result, the present inventors have obtained the findings that it is possible to efficiently produce an adult insect having an abnormality in the wing formation and being incapable of flying by suppressing the expression of the above-mentioned genes. Furthermore, the present inventor have examined in detail the relationship between the suppression of expression of each gene and the wing formation, as well as the relationship between the time of suppression of expression and the wing formation, and they have had significant and interesting findings. Furthermore, the influence (effect) of the suppression of expression of the vestigial gene on the wing formation is confirmed also in experiments using Harmonia axyridis.

The present invention is mainly based on the above-mentioned findings and the first aspect of the present invention provides the following biopesticide.

[1] A biopesticide comprising an insect belonging to family Coccinellidae, the insect having an abnormality in the wing formation by suppression of expression of a vestigial gene and/or a scalloped gene.

[2] The biopesticide described in [1], wherein the suppression of expression is carried out by using an RNAi method targeting the vestigial gene and/or the scalloped gene.

[3] The biopesticide described in [2], wherein the RNAi method is carried out by administering dsRNA.

[4] The biopesticide described in any one of [1] to [3], wherein the suppression of expression is carried out in a larval stage of the insect.

[5] The biopesticide described in any one of [1] to [4], wherein the insect is a ladybug belonging to subfamily Coccinellinae.

[6] The biopesticide described in any of [1] to [4], wherein the insect is Harmonia axyridis.

On the other hand, the second aspect of the present invention provides a producing method of a ladybug useful as a biopesticide, and a ladybug produced by the method.

[7] A producing method of an insect belonging to family Coccinellidae having an abnormality in the wing formation, the method including carrying out suppression of expression of a vestigial gene and/or a scalloped gene.

[8] The producing method described in [7], wherein the suppression of expression is carried out by using an RNAi method targeting the vestigial gene and/or the scalloped gene.

[9] The producing method described in [8], wherein the RNAi method is carried out by administering dsRNA.

[10] The producing method described in any of [7] to [9], wherein the suppression of expression is carried out in a larval stage of the insect.

[11] The producing method described in [7], wherein the suppression of expression is carried out by administering dsRNA targeting the vestigial gene to second instar to fourth instar larvae.

[12] The producing method described in [7] or [11], wherein the suppression of expression is carried out by administering dsRNA targeting the scalloped gene to first instar to second instar larvae.

[13] The producing method described in [7] or [12], wherein the insect is a ladybug belonging to subfamily Coccinellinae.

[14] The producing method described in any of [7] to [12], wherein the insect is Harmonia axyridis.

[15] An insect belonging to family Coccinellidae produced by the producing method described in any of [7] to [14].

### BRIEF DESCRIPTION OF THE DRAWINGS

FIG. 1 schematically shows the forms of larvae of Harmonia axyridis in each growth stage.

FIG. 2 shows a full length cDNA sequence of a vestigial gene (vg) of Henosepilachna vigintioctopunctata and a deduced amino acid sequence encoded thereby.

FIG. 3 shows a partial sequence of cDNA of a scalloped gene (sd) of Henosepilachna vigintioctopunctata and a deduced amino acid sequence encoded thereby.

FIG. 4 shows the results of RNAi (larval RNAi method) experiments with respect to larvae of Henosepilachna vigintioctopunctata. Ev-vg (a-d): phenotypes obtained by injecting the double-stranded RNA of vg into each larval stage, Ev-sd (e-h): phenotypes obtained by injecting the double-stranded RNA of sd into each larval stage, Ev-vg+Ev-sd (i-l): phenotypes obtained by injecting the double-stranded RNA of vg and the double-stranded RNA of sd into each larval stage, and gfp (m-p): phenotypes obtained by injecting the double-stranded RNA of gfp into each larval stage (control).

FIG. 5 shows the results of RNAi (larval RNAi method) experiments with respect to larvae of Harmonia axyridis. Ha-vg: phenotype obtained by injecting the double-stranded RNA of vg into the larval stage; and gfp: phenotype obtained by injecting the double-stranded RNA of gfp into the larval stage (control).

FIG. 6 shows results of RNAi (larval RNAi method) experiments with respect to larva of Harmonia axyridis, showing a phenotype obtained by the double-stranded RNA of sd into the larval stage.

### BEST MODE FOR CARRYING OUT THE INVENTION

The biopesticide of the present invention includes an insect belonging to family Coccinellidae having an abnormality in the wing formation by suppression of expression of a certain gene.

In the present invention, the terms “suppression of expression” is used interchangeably with the terms “inhibition of expression” and “inhibition of function.” Furthermore, the term “ladybug” is used to collectively refer to an insect belonging to family Coccinellidae and the ladybug constituting the biopesticide of the present invention is referred to as the “ladybug of the present invention.”

The “biopesticide” generally refers to pesticides using an organism and is different from a chemical pesticide. Among biopesticides, biopesticides using natural enemy organisms are referred to as “natural enemy pesticides.” The biopesticide of the present invention corresponds to the natural enemy pesticides. The biopesticides generally have a shorter effective period as compared with chemical pesticides due to their essential characteristics. In particular, this is remarkable in the case of biopesticide using insects. The biopesticide of the present invention can achieve the remarkable extension of effective period as compared with the case where a natural ladybug is used. Furthermore, it can efficiently suppress the influence on the ecosystem. That is to say, the great characteristics of the present invention are that a durable effect can be exhibited and high safety is obtained.

An example of the insect belonging to family Coccinellidae includes insects belonging to subfamily Coccinellinae: Harmonia axyridis, Aiolocaria hexaspilota, Propylaea japonica, Coccinella septempunctata, Menochilus sexmaculatus, Vibidia duodecimguttata and Illeis koebelei; insects belonging to Scymninae: Amida tricolor, insects belonging to Chilocorinae: Phymatosternus lewisii, Chilocorus rubidus, and Chilocorus kuwanae; insects belonging to Epilachninae: Epilachna vigintioctopunctata Fabricius, Epilachna vigintioctomaculata Motschulsky and Epilachna admirabilis, and the like. Insects of the biopesticide of the present invention preferably is ladybugs belonging to the subfamily Coccinellinae that is generally a beneficial insect. Specifically, Harmonia axyridis, Aiolocaria hexaspilota, Propylaea japonica, Coccinella septempunctata, Menochilus sexmaculatus, Vibidia duodecimguttata, Illeis koebelei, and the like, are preferable. Harmonia axyridis, Aiolocaria hexaspilota, Propylaea japonica, Coccinella septempunctata and Menochilus sexmaculatus are carnivorous and prey upon aphid, scale insect, and the like. Furthermore, Vibidia duodecimguttata and Illeis koebelei fungivorous and prey on powdery mildew, and the like. Particularly preferably, the insect of the biopesticide of the present invention is Harmonia axyridis.

In the present invention the term “having an abnormality in the wing formation” means that normal wing formation cannot be carried out, thus causing a disorder in the flying ability. The level of “disorder” herein is not particularly limited as long as the flying ability is different from that of the corresponding natural (wild type) ladybug. Preferably, the ladybug of the present invention does not have a substantial flying ability.

The presence or degree of flying ability of a ladybug can be evaluated by measuring, for example, the number of flying time or the flying distance after it is left for a predetermined period of time. When the flying abilities are compared between ladybug to be tested and corresponding wild type ladybug by using the above-mentioned evaluation system, it is possible to determine the degree of disorder of the flying ability of the ladybug to be tested. Specifically, by allowing the ladybug to be tested and the corresponding wild type ladybug to drop under the same conditions, both ladybugs are compared in terms of the presence of flying and/or the flying distance. Thereby, the flying ability of the ladybug to be tested can be evaluated. Note here that a “corresponding ladybug” refers to a ladybug of the same kind. For example, when the ladybug according to the present invention is Harmonia axyridis, the corresponding ladybug is a wild type Harmonia axyridis.

With the above-mentioned test system, the flying ability of a ladybug can be evaluated and determined reliably. On the other hand, the flying ability of a ladybug can be also estimated by observing the form of the wing. For example, as a result of the observation of the forms, when the wing is not observed, or the size of the wing is extremely small and judged that it does not function as a wing, it is possible to evaluate and determine that the ladybug does not substantially have the flying ability.

In the present invention, genes to which the suppression of expression is carried out include a vestigial gene (which is abbreviated as “vg” if necessary in the present specification) and a scalloped gene (which is abbreviated as “sd” if necessary in the present specification). That is to say, a ladybug having an abnormality in the wing formation is produced by suppressing the expression of either or both of these genes, and the obtained ladybug is used as the biopesticide of the present invention.

The vestigial gene is a gene cloned as a master gene of the wing formation in Drosophila. It is thought that the vestigial gene is an essential gene for winged insects. However, due to its poor preservability, success in cloning in other species have been hardly reported. The present inventors group has reported the success in cloning a vestigial gene of Henosepilachna vigintioctopunctata based on the sequence information of the vestigial gene of Drosophila or Culicidae (master's thesis: “Isolation and Function Analysis of Insects Wing Formation Genes,” Masayo Miwa, Graduate School of Bioagricultural Sciences of Nagoya University, Department of Biological Mechanisms and Functions, March 2002). The vestigial gene (cDNA) of Henosepilachna vigintioctopunctata and the amino acid sequence encoded thereby are shown in SEQ ID NO: 1 and SEQ ID NO: 2, respectively. The present inventors have succeeded in cloning of a partial sequence of the vestigial genes of Harmonia axyridis by the same method. The vestigial gene (cDNA) of Harmonia axyridis and the amino acid sequence encoded thereby are shown in SEQ ID NO: 5 and SEQ ID NO: 6, respectively.

The cloning of the vestigial genes of certain ladybugs can be carried out with reference to the above-mentioned report (master's thesis: “Isolation and Function Analysis of Insects Wing Formation Genes,” Masayo Miwa, Graduate School of Bioagricultural Sciences of Nagoya University, Department of Biological Mechanisms and Functions, March 2002) or the below-mentioned Example. Briefly, the intended vestigial genes can be obtained through the extraction of the total RNA from the wing primordium in the larval stage, synthesis of the single-stranded cDNA, and RT-PCR using specific primers.

On the other hand, the scalloped gene is a gene that has been cloned as a gene essential to the wing formation similar to the vestigial gene, and the presence has been confirmed in, for example, Drosophila. It is thought that the scalloped gene is involved in the wing formation in corporation with the vestigial gene. The present inventors group has reported the success in cloning of the scalloped gene (partial sequence) of Henosepilachna vigintioctopunctata (master's thesis: “Isolation and Function Analysis of Insects Wing Formation Genes,” Masayo Miwa, Graduate School of Bioagricultural Sciences of Nagoya University, Department of Biological Mechanisms and Functions, March 2002). The scalloped gene (cDNA partial sequence) of Henosepilachna vigintioctopunctata and the amino acid sequence encoded thereby are shown in SEQ ID NO: 3 and SEQ ID NO: 4, respectively. The present inventors have succeeded in cloning of a partial sequence of the scalloped gene of Harmonia axyridis by the same method. The scalloped gene (cDNA partial sequence) of Harmonia axyridis and the amino acid sequence encoded thereby are shown in SEQ ID NO: 15 and SEQ ID NO: 16, respectively.

Similar to the vestigial gene, the cloning of the scalloped genes of certain ladybugs can be carried out with reference to the above-mentioned report (master's thesis: “Isolation and Function Analysis of Insects Wing Formation Genes,” Masayo Miwa, Graduate School of Bioagricultural Sciences of Nagoya University, Department of Biological Mechanisms and Functions, March 2002) or the below-mentioned Example. Briefly, the intended scalloped genes can be obtained through the extraction of the total RNA from the wing primordium in the larval stage, synthesis of the single-stranded cDNA, and RT-PCR using specific primers.

Methods for suppressing the expression of the vestigial genes and scalloped genes are not particularly limited and can employ, for example, an RNAi method, an antisense method, or a method using ribozyme. In the case of these methods, it is possible to suppress the expression of the target genes without affecting genes in the genome. Thus, it is possible to achieve a biopesticide that does not substantially influence on the ecosystem by inheritance to the next generation. Such characteristics are preferable as biopesticides accompanying insects scatter to outdoors.

### (Suppression of Expression by RNAi Method)

It is particularly preferable that the RNAi method is employed as a method for suppressing the expression. The RNAi method can achieve the specific and effective suppression of expression. The “RNAi” refers to a phenomenon in which the expression of the target gene is suppressed by introducing an RNA having sequences homologous to the target genes (in particular, homologous to mRNA corresponding to the target gene) into the target cells.

In the suppression of expression by the RNAi method using insects, in general, dsRNA (double-stranded RNA) corresponding to a part of the target gene (the vestigial gene or the scalloped gene in the case of the present invention) is used. Two or more dsRNAs may be used with respect to one target gene.

In the case of the RNAi method targeting mammalian cells, short-length dsRNA (siRNA) having about 20 to 23 nucleotides is used. On the other hand, in insect cells, a rather long-length dsRNA having several hundreds of nucleotides or more is effectively used. The length of dsRNA to be used in the RNAi method is, for example, 30 nucleotides or more, and preferably 200 nucleotides or more. In order to cause an effective suppression of expression, dsRNA is preferable, however, a single-stranded RNA may be used.

The dsRNA to be used is not necessarily divided into two molecules, a sense chain and an antisense chain. For example, a sense chain and an antisense chain constituting dsRNA may have a structure in which they are linked to each other by a hairpin loop.

The dsRNA is preferably introduced into the target organism (egg, larva, pupa or adult) by injecting a dsRNA solution. However, as mentioned below, it may be introduced by way of feeding (see, Timmons L, Fire A: Specific Interference by ingested dsRNA. Nature 395: 854, 1988). Furthermore, a method of immersing a larva in a dsRNA solution (see, Tabara H, Grishok A, Mello C C: RNAi in C. elegans: soaking in the genome sequence. Science 282: 430-431, 1998) or a method of immersing an egg in a dsRNA solution may be employed. Regarding the protocol of RNAi of a ladybug, see the research paper of the present inventors (Niimi Teruyuki, et al. RNAi Protocol of Harmonia axyridis, Cell Technology, Vol. 22 No. 1 pp 80-85. 2003).

Instead of or in addition to directly (intact) introducing dsRNA into target organisms, an expression vector into which a DNA sequence encoding the target dsRNA may be introduced into the target organisms. The RNAi using an expression vector in this way permits, for example, long-lasting controlling of the RNAi effect.

The dsRNA used in the RNAi method can be prepared in vitro or in vivo by chemical synthesis or by using an appropriate expression vector. The method using an expression vector is particularly effective in preparing relatively long dsRNA. To design dsRNA, in general, a sequence (continuous sequence) specific to the target nucleic acid is used. Note here that programs and algorithms for selecting appropriate target sequences have been developed.

Specific examples of preparation method of dsRNAs are shown in the column of Example mentioned below.

### (Suppression of Expression by Antisense Method)

When suppression of expression by an antisense method is carried out, the method generally uses an antisense construct which produces specific RNA complementary to mRNA corresponding to target gene when it is transcribed. Such an antisense construct (also referred to as “antisense nucleic acid”) is introduced into the target cells in the form of an expression plasmid. When the form as the antisense construct is introduced in the target cells, oligonucleotide probe hybridized to a DNA sequence of the target gene or the mRNA sequence corresponding to the DNA sequence (which are also collectively referred to as “target nucleic acid”) and inhibiting thereof can be employed. As such an oligonucleotide probe, one having a resistivity with respect to an endogenous nuclease such as exonuclease and/or endonuclease can be preferably used.

When DNA molecule is used as the antisense nucleic acid, oligodeoxyribo nucleotide derived from a region including a translation initiation site (for example, region from −10 to +10) of mRNA corresponding to the target cells is preferred.

It is preferable that the complementation between the antisense nucleic acid and the target nucleic acid is strict, however, somewhat mismatch may be accepted. The hybridization ability of the antisense nucleic acid to the target nucleic acid is generally dependent upon both the degree and length of complementation of both nucleic acids. In general, as the length of the antisense nucleic acid to be used is longer, stable double strand (or triple strand) with the target nucleic acid can be formed even if the number of mismatches is larger. A person skilled in the art can confirm the degree of acceptable mismatches by using standard techniques.

The antisense nucleic acid may be DNA, RNA or chimera mixtures, or derivatives or modified products thereof. Furthermore, it may be single-stranded or double-stranded. The stability and hybridization ability and the like of the antisense nucleic acid can be improved by modifying the base portion, sugar portion or phosphoric acid skeleton portion.

The antisense nucleic acid can be synthesized by a routine procedure by using, for example, a commercially available automated DNA synthesizer (for example, product of Applied Biosystems). For producing modified nucleic acid or derivatives, see, for example, Stein et al. (1988), Nucl. Acids Res. 16:3209 and Sarin et al., (1988), Proc. Natl. Acad. Sci. U.S.A. 85:7448-7451.

In order to enhance the operation of the antisense nucleic acid in the target cells, a promoter (for example, an actin promoter, ie1 promoter, and the like) that strongly acts in insects cells can be used. That is to say, when a construct including antisense nucleic acid disposed under the control of such promoters is introduced in target cells, the transcription of sufficient amount of antisense nucleic acid can be secured due to the action of the promoter.

### (Suppression of Expression by Ribozyme)

Another embodiment of the present invention relates to suppression of expression by ribozyme. The mRNA corresponding to the target gene may be destroyed by using ribozyme capable of cleaving mRNA at the site-specific recognition sequence. Preferably, hammerhead ribozyme is used. As to the method for constructing the hammerhead ribozyme, see, for example, Haseloff and Gerlach, 1988, Nature, 334:585-591.

Similar to the antisense method, for example, for the purpose of improving the stability and target property, ribozyme may be constructed by using modified oligonucleotide. In order to allow the effective amount of ribozyme to generate in the target cells, under the control of a promoter strongly acting in the insect cells (for example, actin promoter and ie1 promoter), a nucleic acid construct including DNA encoding the ribozyme is preferably used.

As shown in the below-mentioned Example, the present inventors have investigated to clarify the relationship between the suppression of expression of each gene and the wing formation, and the relationship between the time of suppression of expression and the wing formation. Based on the findings, in one embodiment of the present invention, by suppressing the expression of the vestigial gene in larva in the second to fourth instar, preferably in the third to fourth instar, furthermore preferably in the early third to the early fourth instar, and most preferably in the late third instar (when a method for suppressing the expression by an RNAi method is employed, for example, dsRNA targeting the vestigial gene is administered), a ladybug having an abnormality in the wing formation is obtained. In another exemplary embodiment of the present invention, the similar suppression of expression targeting the scalloped gene is carried out to larva in the first to second instar, and preferably larva in the first instar. Furthermore, when suppression of expression of the vestigial gene and scalloped gene is carried out, the similar operation is carried out to larva in the first to second instar, and preferably larva in the first instar.

Note here that in this specification, “time of the suppression of expression” refers to “time at which an operation of the suppression of expression is carried out.” Therefore, when the RNAi method using injection is carried out, the injection time is the “time of suppression of expression.” Therefore, the time at which the effect of the suppression of expression occurs in the organism is later than time of the suppression of expression.

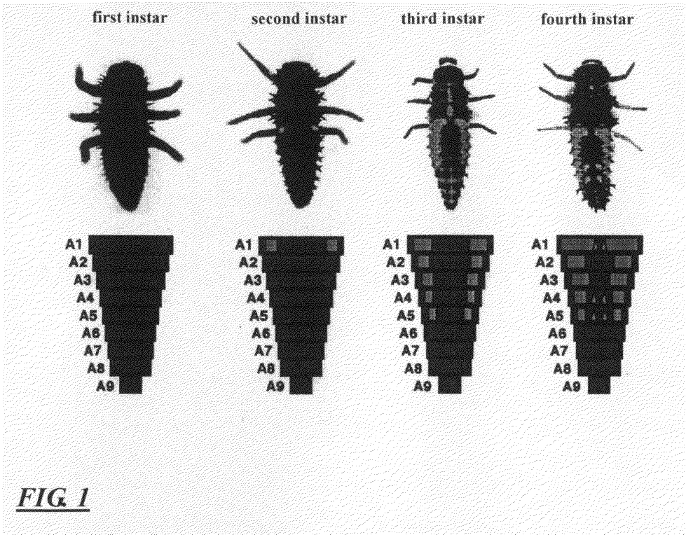

The larval stage of a ladybug is divided into the first instar, second instar, third instar, and fourth instar. Hereinafter, each instar is described taking larva of Harmonia axyridis shown in FIG. 1 as an example. The first instar is a period right after incubation and lasts about two to four days. The larva in the first instar has a short body length and a body color of black. The second instar is a period following the first instar and a period in which patterns occur in the segment. The length of this period is typically about two to four days. When orange-colored spots are present in the segment A1, the larva in the second instar can be identified. After the second instar, the larva enters the third instar. Spots appear in the peripheral portions of the segments A1 to A5. The third instar generally lasts about two to four days. The fourth instar is a last period of the larval stage. This period is a preparation period from larva to pupa. The larva in the fourth instar has a large body length and have spots in the peripheral portions from the segments A1 to A5. In the segments A1, A4 and A5, spots appear also around the center.

Note here that in this specification, the early one to two days are referred to as an early stage and the late one to two days are referred to as a late stage.

By suppressing the expression by the injection to larva as mentioned above, unlike the genetic modification operation by transformant, basically, the next generation is not genetically affected. Therefore, it is said that the influence of the biopesticide of the present invention on the ecosystem is extremely small.

When the expression suppression in larva is carried out by the RNAi method, typically, a solution containing dsRNA is injected into larva. The injection site is not particularly limited. For example, the solution may be injected into the intersegmental membrane in the thorax by using, for example, a glass needle. The injection amount (liquid amount) is set in the range that does not affect the existence of the larva. For example the injection amount may be 0.2 μl to 0.5 μl. The amount of dsRNA to be introduced is an amount that is necessary to obtain the expected expression effect and that does not affect the existence of the larva. It may be about 0.4 μg to 1 μg although it varies depending upon the base length. When the expression of both the vestigial gene and the scalloped gene is suppressed, at least two kinds of dsRNA are used. In this case, the amount is determined so that the total RNA amount falls within in the above-mentioned range.

Note here that the method for causing RNAi by injection operation with respect to larva is also referred to as “larval RNAi method” in this specification. The larval RNA method directed to a ladybug is described in, for example, T. Niimi, H. Kuwayama and T. Yaginuma: Larval RNAi Applied to the Analysis of Postembryonic Development in the Ladybug beetle, Harmonia axyridis. Journal of Insect Biotechnology and Sericology 74, 95-102(2005), and Niimi Teruyuki, Yanaginuma Toshinobu (2006) Harmonia axyridis, larval RNAi method, news of the Japan Society for Comparative Endocrinology, 121, 32-37.

In one embodiment of the present invention, the suppression of expression of the target gene is carried out with respect to female adult insects so as to allow the next generation created from the female adult insects to have an abnormality in the wing formation. In the RNAi method for example, a predetermined dsRNA is injected into a female adult insect. The injection site is an abdomen in which the genital organ is present. Note here that the RNAi method directed to the adult insect is referred to as “Parental RNAi method” in this specification. In the larval RNAi method, injection operation is required to be carried out individually per one larva. According to the Parental RNAi method, however, by carrying out the injection into one individual female adult insect, a large number of ladybugs having an abnormality in the wing formation can be obtained.

In another embodiment of the present invention, the suppression of expression is carried out via feeding. Specifically, for example, siRNA to a target gene is mixed in artificial feed and the mixture is fed to a larva. Such suppression of expression via feeding makes it possible to obtain the objective ladybug, that is, a ladybug having an abnormality in the wing formation in a simple way.

### Example

### <Production of Henosepilachna Vigintioctopunctata Having Abnormality in Wing Formation>

1. Cloning of Vestigial Gene (vg) and Scalloped Gene (sd)

According to the aforementioned report (master's thesis: “Isolation and Function Analysis of Insects Wing Formation Genes,” Masayo Miwa, Graduate School of Bioagricultural Sciences of Nagoya University, Department of Biological Mechanisms and Functions, March 2002), cDNA (partial sequence) of a vestigial gene (vg) and cDNA (partial sequence) of a scalloped gene (sd) are cloned by the below-mentioned method. Note here that this report describes the cloning of cDNAs (partial sequence) of vg homologue and sd homologue from several kinds of insects including Henosepilachna vigintioctopunctata and the determination of the base sequence and the deduced amino acid sequence thereof.

### (1) Experimental Insects

Henosepilachna vigintioctopunctata (conventional nomenclature: Epilachna vigintioctopunctata) (collected from potato leaves in the cultivated land in Graduate School of Bioagricultural Sciences of Nagoya University) was used.

(2) Preparation of cDNA

A total RNA was extracted from a wing primordium of larva in a prepupa stage of Henosepilachna vigintioctopunctata (Ev) by a guanidine hydrochloride method using TRIZOL (Invitrogen). This RNA was used as a template to synthesize a single-stranded cDNA by using Superscript II reverse transcriptase (Invitrogen) based on SMART™ RACE cDNA Amplification Kit (CLONTECH).

### (3) RT-PCR

Polymerase chain reaction (PCR) was carried out by using the single-stranded cDNA prepared as mentioned above as a template. For Taq DNA polymerase of PCR, Ampli Taq Gold (Applied Biosystems) was used. Primers used in PCR are shown below. Note here that in the sequences, W represents A+T, S represents C+G, Y represents C+T, R represents A+G, and I represents inosine, respectively.

vg sense primer;
vg-01:

(23mer, SEQ ID NO: 7)

5′-GTIWSITGYCCIGARGTIATGTA-3′
vg antisense primer;
vg-04:

(24mer, SEQ ID NO: 8)

5′-RTAYTGIGCCATRTTRTGRTGRTA-3′
sd sense primer;
sd-01:

(18mer, SEQ ID NO: 9)

5′-GAYGCIGARGGIGTITGG-3′
sd antisense primer;
sd-04:

(26merm SEQ ID NO: 10)

5′-TTYTCIARIACISWRTTCATCATRTA-3′

The followings are conditions of the PCR.

(a) vg cDNA

Activation reaction of enzyme: 95° C., 9 minutes

Cycle: 50

Denaturation reaction: 94° C., 1 minute

Annealing reaction: 49° C., 30 seconds

Elongation reaction: 72° C., 3 minutes

(b) sd cDNA

Activation reaction of enzyme: 95° C., 9 minutes

Cycle: 50

Denaturation reaction: 94° C., 1 minute

Annealing reaction: 41° C., 30 seconds

Elongation reaction: 72° C., 2 minutes

(4) Subcloning of PCR Product into pBluescript

Each RT-PCR product as an insert was inserted into an Eco RV recognition site of pBluescript™KS (+) (pBS). The ligation reaction was carried out by using DNA Ligation Kit Ver. 2 (TaKaRa). This ligation reaction solution was used for transformation to Escherichia coli (XL1-Blue). This transformed Escherichia coli culture solution was plated on an LB plate and cultured at 37° C. overnight. White colony was screened to select a clone into which the objective PCR fragment was inserted by using a PCR method. For Taq DNA polymerase for PCR at this time, SIGMA Taq (SIGMA) was used. As the primer, an SK primer and a KS primer were used. PCR was carried out 25 cycles. The cycle includes denaturing at 95° C. for 30 seconds; annealing at 55° C. for 30 seconds; and elongation at 72° C. for 30 seconds. The selected clone was subjected to shaking culture at 37° C. and thus plasmid DNA was prepared by using FlexiPrep Kit (Amersham Biosciences) according to the protocol of the kit.

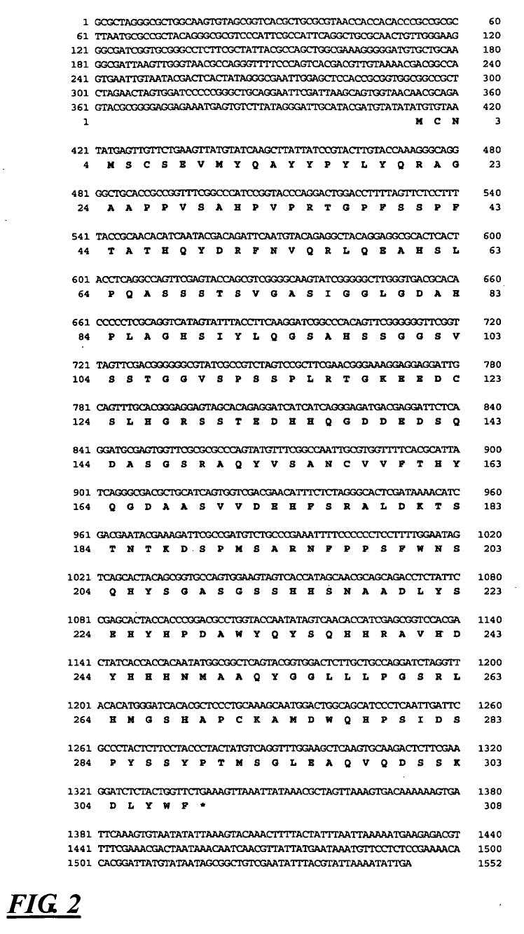

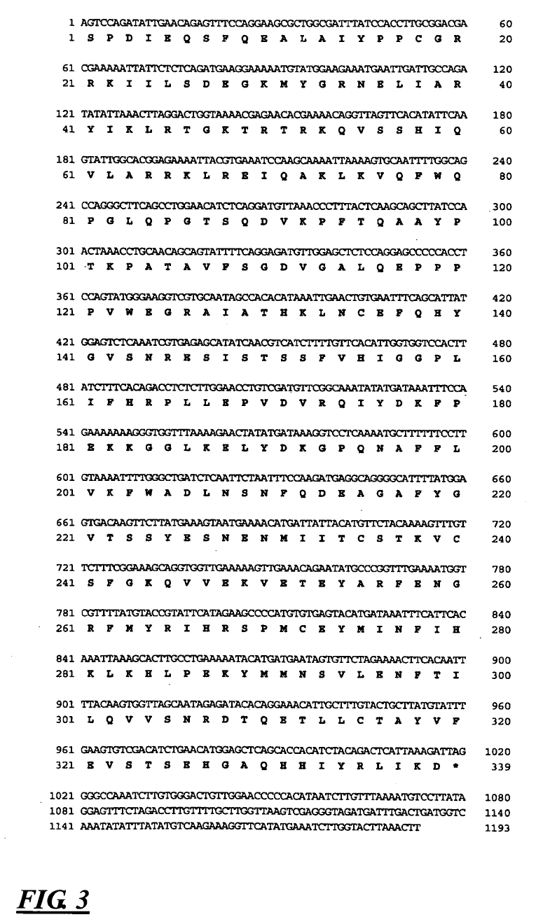

As mentioned above, pBluescript KS+ in which an RT-PCR product amplified with vg-01 and vg-04 (vg cDNA partial sequence) was subcloned to an EcoRV site, and pBluescript KS+ in which an RT-PCR product amplified with sd-01 and sd-04 (sd cDNA partial sequence) was subcloned to an EcoRV site were obtained. The full-length cDNA base sequence of vg (SEQ ID NO: 1) and the deduced amino acid sequence (SEQ ID NO: 2) are shown in FIG. 2. In FIG. 2, sequence at base number 445-1141 corresponds to the subcloned cDNA partial sequence of vg. On the other hand, cDNA partial sequence of sd (SEQ ID NO: 3) and the deduced amino acid sequence corresponding thereto (SEQ ID NO: 4) are shown in FIG. 3. In FIG. 3, sequence at base number 1-864 corresponds to the subcloned cDNA partial sequence of sd.

2. Effect of Suppression of Expression of vg and/or sd by Larval RNAi Method

### (1) Amplification of Template for Synthesizing RNA

As a template for synthesizing double-stranded RNA, a PCR product to which a promoter sequence of T7 RNA polymerase had been added on both terminals of the cloned genes (vg cDNA partial sequence and sd cDNA partial sequence) were used. When PCR is carried out, the following PCR primer was used. In the PCR primer, a promoter sequence of T7 RNA polymerase is added to a vector sequence so that it can be commonly used for any genes cloned to the above-mentioned vector.

T7-KS primer:

(SEQ ID NO: 11)

5′-TAATACGACTCACTATAGGGAGACCACTCGAGGTCGACGGTATC-3′
T7-SK primer:

(SEQ ID NO: 12)

5′-TAATACGACTCACTATAGGGAGACCACCGCTCTAGAACTAGTGGA
TC-3′

The PCR was carried out under the following conditions. Four to eight of the following reaction tubes were prepared to obtain the sufficient amount of PCR product.

### (a) Reaction Solution

Template DNA (20 to 50 ng)+H₂O: 37.75 μl

10×Buffer: 5 μl

2 mM dNTP: 5 μl

10 pmol/ml T7-KS primer: 1 μl

10 pmol/ml T7-SK primer: 1 μl

AmpliTaq Gold (Applied Biosystems): 0.25 μl

Total: 50 μl

### (b) Reaction Conditions

The PCR was carried out in the following conditions. One cycle includes: 1st stage: 95° C. for 9 minutes, 2nd stage: 94° C. for 60 seconds, 55° C. for 30 seconds, and 72° C. for 60 seconds per cycle. This cycle was repeated 40 cycles. 3rd stage: 72° C. for 7 minutes. 4th stage: 4° C. ∞.

The PCR product obtained by the above-mentioned reaction was concentrated by ethanol precipitation, and subjected to agarose electrophoresis. Then, template DNA was purified from gel by using Mag Extractor (Toyobo). In general, the amount of the obtained template DNA corresponds to the several times of synthesizing reaction of double-stranded RNA mentioned below.

### (2) Synthesis of Double-Stranded RNA

The above-mentioned template DNA (1 μg) was used to synthesize RNA according to MEGAscript T7 Kit (Ambion). The RNA was dissolved in an appropriate amount of nuclease free pure water.

In order to anneal double-stranded RNA, the obtained RNA solution was incubated at 65° C. for 30 minutes by using a heat block, and then returned to room temperature over 1 to 2 hours. A small amount of this double-stranded RNA was confirmed by concentration measurement and confirmation by agarose electrophoresis. The double-stranded RNA that had been synthesized was divided into small parts so that each part corresponds to one injection and stored at −80° C.

(3) Injection into Larval Body

By using an injection device using FemtoJet (Eppendorf), an appropriate amount of double-stranded RNA was injected to larvae (first instar larva, early third instar larva, late third instar larva, and early fourth instar larva) anesthetized with carbon dioxide. As the injector, a glass tube (GDC-1, Narishige) with filament having an outer diameter of 1 mm was used. For each larva, about 0.5 μl (about 0.5 μg) of double-stranded RNA was injected at the concentration of about 1 μg/μl. When the double-stranded RNA of vg and double-stranded RNA of sd were injected at the same time, the mixture solution whose injection amount was adjusted to about 0.25 μg each was used.

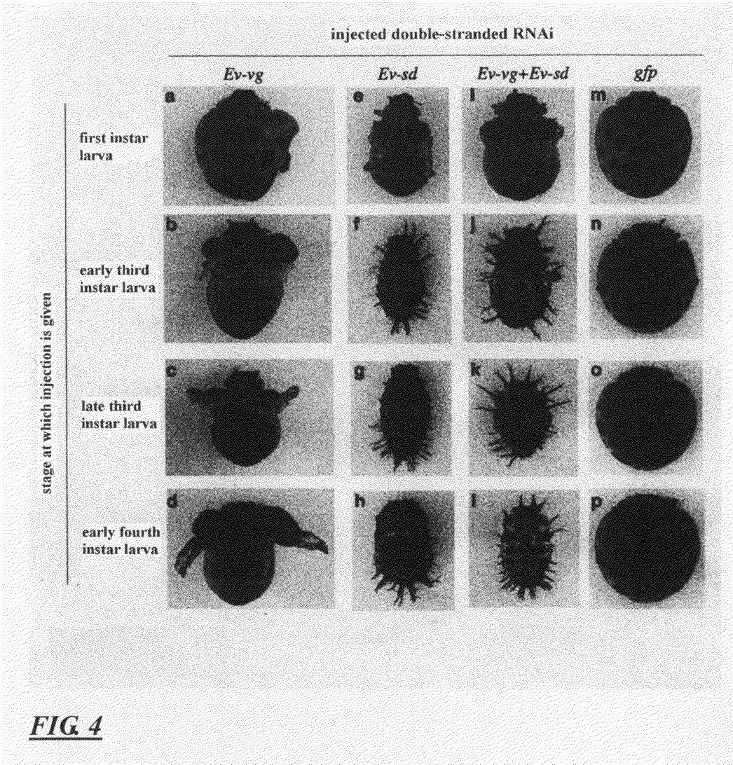

Larvae after injection operation were reared under the normal conditions and subjected to morphological observation at the stage when the larva became an adult insect. The results of the morphological observation are shown in FIG. 4. Reference marks “a” to “d” show phenotypes (adult) obtained by injecting the double-stranded RNA of vg into each larval stage (the injection time is the first instar, early third instar, late third instar larva, and early fourth instar larva in this order). In “a”, some wing deformation is observed. In “b” to “d”, remarkable deformation and contraction of the wing were observed, showing that the flying ability is seriously damaged.

On the other hand, reference mark “e” shows a phenotype (adult) obtained by injecting the double-stranded RNA of sd into the first instar larva, showing only a sign of a wing and completely lacking the flying ability. When the double-stranded RNA of vg and double-stranded RNA of sd are injected, (i) also lacks the flying ability.

Reference marks “f” to “h” (the case where the double-stranded RNA of sd is injected into early third instar larva, late third instar larva, and early fourth instar larva) and reference marks “j” to “l” (when the double-stranded RNAs of vg and sd are injected into early third instar larva, late third instar larva, and early fourth instar larva), showing that the insects die immediately before pupation and do not grow into adult insects.

From the above-mentioned results, the following findings are derived.

(a) Both RNAi targeting vg and RNAi targeting sd can substantially affect the wing formation. In other words, with these methods, an adult insect having an abnormal flying ability or lacking the flying ability can be obtained.

(b) The influence on the wing formation (effect of RNAi) is largely different depending upon the injection time of the double-stranded RNAi. That is to say, when the vg is targeted, the effect of the injection to young instar larvae is low. In particular, the severe effect cannot be expected when the injection is carried out to the first instar larva. It is thought to be preferable that the injection is carried out in the second instar or later. In particular, the severe effect can be expected when the injection is carried out with respect to the third instar larva to the fourth instar larva. Among them, the most effective time is the late third instar.

(c) When sd is targeted, it is preferable that the injection is carried out to the young instar larva. Specifically, it is preferable that the injection is carried out to larva before the third instar. In particular, it is thought to be preferable that the injection is carried out to the first instar. By injecting to the first instar larva, an adult insect completely lacking the flying ability can be obtained. On the other hand, when the injection time is late, lethal influence may occur.

(d) The case in which both vg and sd are targeted is the same as the case in which sd is targeted, with the injection at young instar (in particular, injection at the first instar is preferable), adult completely lacking the flying ability can be obtained.

As mentioned above, as the producing method of an adult ladybug having an abnormality (damaged flying ability) in the wing formation, a larval RNAi method targeting vg and/or sd is shown to be effective. Furthermore, it is determined that the injection time (time at which RNAi operation is carried out) is important. The time at which the wing formation can be effectively inhibited is clarified.

### <Production of Harmonia Axyridis Having Abnormality in Wing Formation>

The vestigial gene (vg) of Harmonia axyridis (using Harmonia axyridis collected outdoor and subcultured) was cloned by the same procedures (1. (1) to (4)) in the case of Henosepilachna vigintioctopunctata mentioned above except that fore wing primordium of pupa one day after pupation was used as samples for preparing cDNA and the primer sets used for RT-PCR were the following vg5 to vg7. Note here that W represents A+T, S represents C+G, Y represents C+T, R represents A+G, and I represents inosine, respectively.

vg sense primer;
vg-05:

(29mer, SEQ ID NO: 13)

5′-ATGTAYSRIGCITAYTAYCCITAYYTITA-3′
vg antisense primer;
vg-07:

(26mer, SEQ ID NO: 14)

5′-SWRTTCCARAAISWIGGIGGRAARTT-3′

The cDNA partial sequence of successfully cloned vg of Harmonia axyridis and the deduced amino acid sequence corresponding thereto are shown in SEQ ID NO: 5 and SEQ ID NO: 6, respectively. By using the thus prepared cDNA sequence, the double-stranded RNA of vg was synthesized and the effect of suppression of the expression of vg by the Larval RNAi method was examined. Note here that the Larval RNAi method was also carried out as in the case of Henosepilachna vigintioctopunctata.

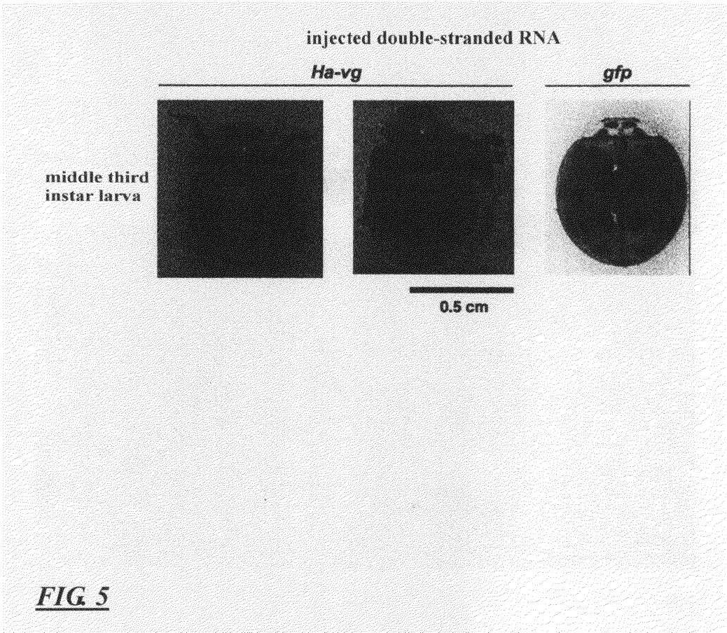

The results of the morphological observation of forms are shown in FIG. 5. Left and middle pictures show phenotypes (adult) obtained by injecting double-stranded RNA of vg to the middle third instar larva. Remarkable deformation and contraction of the wing are observed, showing that the flying ability is seriously damaged. From the results, RNAi targeting vg can substantially affect the wing formation.

The scalloped gene of Harmonia axyridis was cloned by the same procedures (1. (1) to (4)) in the case of Henosepilachna vigintioctopunctata. However, fore wing primordium of pupa one day after pupation was used as sample. Primer sets to be used for RT-PCR are shown below. Note here that the Larval RNAi method was carried out by the same method as in the case of Henosepilachna vigintioctopunctata.

sd sense primer;
sd-01:

(18mer, SEQ ID NO: 9)

5′-GAYGCIGARGGIGTITGG-3′
sd antisense primer;
sd-04:

(26mer, SEQ ID NO: 10)

5′-TTYTCIARIACISWRTTCATCATRTA-3′

The cDNA partial sequence of successfully cloned sd of Harmonia axyridis and the deduced amino acid sequence corresponding thereto are shown in SEQ ID NO: 15 and SEQ ID NO: 16 in the sequence listing, respectively. By using the thus prepared cDNA sequence, the double-stranded RNA of sd was synthesized and the effect of the suppression of expression of sd by the Larval RNAi method was examined. Note here that the Larval RNAi method was carried out in the same manner as in the case of Henosepilachna vigintioctopunctata.

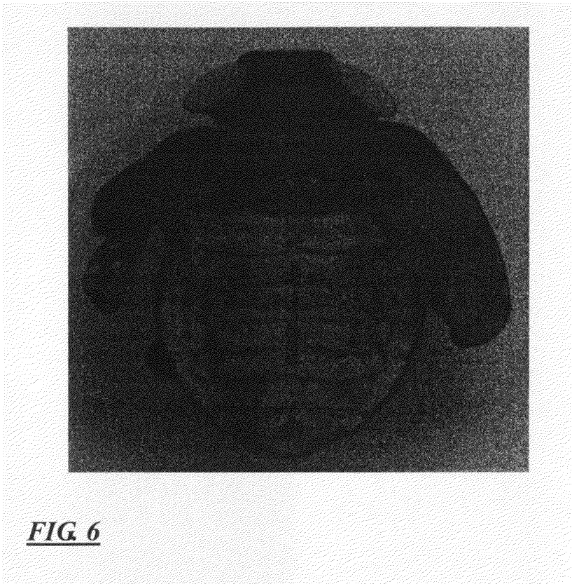

The results of the morphological observation are shown in FIG. 6, showing remarkable deformation and contraction of the wing. Thus, RNAi targeting sd substantially affects the wing formation.

As mentioned above, also in Harmonia axyridis, as the production method of an adult ladybug having an abnormality in the wing formation (damaged flying ability), the larval RNAi method targeting vg or sd is shown to be effective.

### INDUSTRIAL APPLICABILITY

The present invention is used for control methods of insect pest whose natural enemy insect is a ladybug. A ladybug constituting the biopesticide of the present invention has a disorder in the flying ability. Therefore, according to the biopesticide of the present invention, the dispersion of adult insect by flying can be effectively suppressed and efficient and durable effect of insect pest control can be achieved.

The present invention is not limited to the description of the above exemplary embodiments and Examples. A variety of modifications, which are within the scopes of the following claims and which are easily achieved by a person skilled in the art, are included in the present invention. Contents of the theses, Publication of Patent Applications, Patent Publications, and other published documents referred to in this specification are herein incorporated by reference in its entity.

1. A biopesticide comprising an insect belonging to family Coccinellidae, the insect having an abnormality in wing formation by suppression of expression of a vestigial gene and/or a scalloped gene.

2. The biopesticide according to claim 1, wherein the suppression of expression is carried out by using an RNAi method targeting the vestigial gene and/or the scalloped gene.

3. The biopesticide according to claim 2, wherein the RNAi method is carried out by administering dsRNA.

4. The biopesticide according to claim 1, wherein the suppression of expression is carried out in a larval stage of the insect.

5. The biopesticide according to claim 1, wherein the insect is a ladybug belonging to subfamily Coccinellinae.

6. The biopesticide according to claim 1, wherein the insect is Harmonia axyridis.

7. A producing method of an insect belonging to family Coccinellidae having an abnormality in wing formation, the method comprising carrying out suppression of expression of a vestigial gene and/or a scalloped gene.

8. The producing method according to claim 7, wherein the suppression of expression is carried out by using an RNAi method targeting the vestigial gene and/or the scalloped gene.

9. The producing method according to claim 8, wherein the RNAi method is carried out by administering dsRNA.

10. The producing method according to claim 7, wherein the suppression of expression is carried out in a larval stage of the insect.

11. The producing method according to claim 7, wherein the suppression of expression is carried out by administering dsRNA targeting the vestigial gene to second instar to fourth instar larvae.

12. The producing method according to claim 7, wherein the suppression of expression is carried out by administering dsRNA targeting the scalloped gene to first instar to second instar larvae.

13. The producing method according to claim 7, wherein the insect is a ladybug belonging to subfamily Coccinellinae.

14. The producing method according to claim 7, wherein the insect is Harmonia axyridis.

15. An insect belonging to family Coccinellidae produced by the producing method according to claim 7.


SEQUENCE LISTING (16 sequences)

SEQ ID NO: 1 - DNA, 1552 bp, Henosepilachna vigintioctopunctata
gcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattggagctccaccgcggtggcggccgctctagaactagtggatcccccgggctgcaggaattcgattaagcagtggtaacaacgcagagtacgcggggaggagaaatgagtgtcttatagggattgcatacgatgtatatatgtgtaatatgagttgttctgaagttatgtatcaagcttattatccgtacttgtaccaaagggcaggggctgcaccgccggtttcggcccatccggtacccaggactggaccttttagttctccttttaccgcaacacatcaatacgacagattcaatgtacagaggctacaggaggcgcactcactacctcaggccagttcgagtaccagcgtcggggcaagtatcgggggcttgggtgacgcacaccccctcgcaggtcatagtatttaccttcaaggatcggcccacagttcggggggttcggttagttcgacggggggcgtatcgccgtctagtccgcttcgaacgggaaaggaggaggattgcagtttgcacgggaggagtagcacagaggatcatcatcagggagatgacgaggattctcaggatgcgagtggttcgcgcgcccagtatgtttcggccaattgcgtggttttcacgcattatcagggcgacgctgcatcagtggtcgacgaacatttctctagggcactcgataaaacatcgacgaatacgaaagattcgccgatgtctgcccgaaattttcccccctccttttggaatagtcagcactacagcggtgccagtggaagtagtcaccatagcaacgcagcagacctctattccgagcactaccacccggacgcctggtaccaatatagtcaacaccatcgagcggtccacgactatcaccaccacaatatggcggctcagtacggtggactcttgctgccaggatctaggttacacatgggatcacacgctccctgcaaagcaatggactggcagcatccctcaattgattcgccctactcttcctaccctactatgtcaggtttggaagctcaagtgcaagactcttcgaaggatctctactggttctgaaagttaaattataaacgctagttaaagtgacaaaaaagtgattcaaagtgtaatatattaaagtacaaacttttactatttaattaaaaatgaagagacgttttcgaaacgactaataaacaatcaacgttattatgaataaatgttcctctccgaaaacacacggattatgtataatagcggctgtcgaatatttacgtattaaaatattga

SEQ ID NO: 2 - protein, 308 aa, Henosepilachna vigintioctopunctata
MCNMSCSEVMYQAYYPYLYQRAGAAPPVSAHPVPRTGPFSSPFTATHQYDRFNVQRLQEAHSLPQASSSTSVGASIGGLGDAHPLAGHSIYLQGSAHSSGGSVSSTGGVSPSSPLRTGKEEDCSLHGRSSTEDHHQGDDEDSQDASGSRAQYVSANCVVFTHYQGDAASVVDEHFSRALDKTSTNTKDSPMSARNFPPSFWNSQHYSGASGSSHHSNAADLYSEHYHPDAWYQYSQHHRAVHDYHHHNMAAQYGGLLLPGSRLHMGSHAPCKAMDWQHPSIDSPYSSYPTMSGLEAQVQDSSKDLYWF

SEQ ID NO: 3 - DNA, 1193 bp, Henosepilachna vigintioctopunctata
agtccagatattgaacagagtttccaggaagcgctggcgatttatccaccttgcggacgacgaaaaattattctctcagatgaaggaaaaatgtatggaagaaatgaattgattgccagatatattaaacttaggactggtaaaacgagaacacgaaaacaggttagttcacatattcaagtattggcacggagaaaattacgtgaaatccaagcaaaattaaaagtgcaattttggcagccagggcttcagcctggaacatctcaggatgttaaaccctttactcaagcagcttatccaactaaacctgcaacagcagtattttcaggagatgttggagctctccaggagcccccacctccagtatgggaaggtcgtgcaatagccacacataaattgaactgtgaatttcagcattatggagtctcaaatcgtgagagcatatcaacgtcatcttttgttcacattggtggtccacttatctttcacagacctctcttggaacctgtcgatgttcggcaaatatatgataaatttccagaaaaaaagggtggtttaaaagaactatatgataaaggtcctcaaaatgcttttttccttgtaaaattttgggctgatctcaattctaatttccaagatgaggcaggggcattttatggagtgacaagttcttatgaaagtaatgaaaacatgattattacatgttctacaaaagtttgttctttcggaaagcaggtggttgaaaaagttgaaacagaatatgcccggtttgaaaatggtcgttttatgtaccgtattcatagaagccccatgtgtgagtacatgataaatttcattcacaaattaaagcacttgcctgaaaaatacatgatgaatagtgttctagaaaacttcacaattttacaagtggttagcaatagagatacacaggaaacattgctttgtactgcttatgtatttgaagtgtcgacatctgaacatggagctcagcaccacatctacagactcattaaagattaggggccaaatcttgtgggactgttggaacccccacataatcttgtttaaaatgtccttataggagtttctagaccttgttttgcttggttaagtcgagggtagatgatttgactgatggtcaaatatatttatatgtcaagaaaggttcatatgaaatcttggtacttaaactt

SEQ ID NO: 4 - protein, 339 aa, Henosepilachna vigintioctopunctata
SPDIEQSFQEALAIYPPCGRRKIILSDEGKMYGRNELIARYIKLRTGKTRTRKQVSSHIQVLARRKLREIQAKLKVQFWQPGLQPGTSQDVKPFTQAAYPTKPATAVFSGDVGALQEPPPPVWEGRAIATHKLNCEFQHYGVSNRESISTSSFVHIGGPLIFHRPLLEPVDVRQIYDKFPEKKGGLKELYDKGPQNAFFLVKFWADLNSNFQDEAGAFYGVTSSYESNENMIITCSTKVCSFGKQVVEKVETEYARFENGRFMYRIHRSPMCEYMINFIHKLKHLPEKYMMNSVLENFTILQVVSNRDTQETLLCTAYVFEVSTSEHGAQHHIYRLIKD

SEQ ID NO: 5 - DNA, 514 bp, Harmonia axyridis
tcgggctgacatcggcgaatctttcgcatgtgccgacgttttatctaaagcttttgaaaaatgttcatctaccaccgatgccgcatcgccctgataatgggtgaaaaccacgcagttggccgatacatattgcgctcgtgagccactgggttcttgggaatcctcgtcgtcaccttggtgatgatcgtctgtgctactccttccgtgcaaactgcaatcttcttctttccctatcctcagcgggctggagggtgacactcctcccgtcgaactgaccaaacctccagaactatgtgctgacccttgaaggtatatactgtgacccgccagcgggtgagtttctgcgagtcctccgatcgtagcaccgacgctactcgtcgcactcgtctgtggtaaggagtgtgcttcttgaagtctttgtacattgaatctttcatattgatgtgtggcagcaaaaggtgaactaaaaggtccggtcctgggtaccggatgggctgaaacaggtggagcagcc

SEQ ID NO: 6 - protein, 171 aa, Harmonia axyridis
AAPPVSAHPVPRTGPFSSPFAATHQYERFNVQRLQEAHSLPQTSATSSVGATIGGLAETHPLAGHSIYLQGSAHSSGGLVSSTGGVSPSSPLRIGKEEDCSLHGRSSTDDHHQGDDEDSQEPSGSRAQYVSANCVVFTHYQGDAASVVDEHFSKALDKTSAHAKDSPMSAR

SEQ ID NO: 7 - DNA, 23 bp, Artificial Sequence
  sense primer vg-01
gtnwsntgyccngargtnatgta

SEQ ID NO: 8 - DNA, 24 bp, Artificial Sequence
  anti-sense primer vg-04
rtaytgngccatrttrtgrtgrta

SEQ ID NO: 9 - DNA, 18 bp, Artificial Sequence
  sense primer sd-01
gaygcngarggngtntgg

SEQ ID NO: 10 - DNA, 26 bp, Artificial Sequence
  anti-sense primer sd-04
ttytcnarnacnswrttcatcatrta

SEQ ID NO: 11 - DNA, 44 bp, Artificial Sequence
  primer T7-KS
taatacgactcactatagggagaccactcgaggtcgacggtatc

SEQ ID NO: 12 - DNA, 47 bp, Artificial Sequence
  primer T7-SK
taatacgactcactatagggagaccaccgctctagaactagtggatc

SEQ ID NO: 13 - DNA, 29 bp, Artificial Sequence
  sense primer vg-05
atgtaysrngcntaytayccntayytnta

SEQ ID NO: 14 - DNA, 26 bp, Artificial Sequence
  anti-sense primer vg-07
swrttccaraanswnggnggraartt

SEQ ID NO: 15 - DNA, 876 bp, Harmonia axyridis
agtccagatattgaacagagctttcaagaagctttagcaatatatcctccttgtggcaggaggaaaattattctttcagatgaaggaaaaatgtatggaaggaatgaactcattgcaagatacataaaactgcgtactggaaaaacgagaacacgaaagcaggttagttcccatattcaagttttggctcgaagaaaattacgtgaaattcaagccaaactcaaagtgcaattctggcagcctggcttacaaccagggacatcccaagatgtcaaaccctttacacaagcagcttatccaacaaaacctgcaactgctgtgtcgtctggtgaagttggagctcttcaagagcctccacctccagtttgggaaggtcgtgcaatagctacacataaattaagacttgttgaattttctgcatttatggagtctcgaaatcgtgaagaagcatatcaacgtcatctttttgttcacataggtggacctgctctatcatattcagatcctttattagaagctgttgatgttcggcaaatatatgataaatttccagaaaagaaaggtggtttaaaagaattatatgataaaggtcctcaaaatgccttcttccttgtaaaattttgggctgatctcaattctaattttcaagatgaggcgggtgcattttatggtgttacaagttcatatgaaagtaatgaaaatatgattattacgtgctctacaaaagtttgttcttttggaaaacaagttgttgaaaaagttgaaacagaatatgctagatttgaaaatggccgttttgtatacaggattcataggagtccaatgtgtgaatatatgataaatttcattcataaactgaaacacttgcctgagaaa

SEQ ID NO: 16 - protein, 292 aa, Harmonia axyridis
SPDIEQSFQEALAIYPPCGRRKIILSDEGKMYGRNELIARYIKLRTGKTRTRKQVSSHIQVLARRKLREIQAKLKVQFWQPGLQPGTSQDVKPFTQAAYPTKPATAVSSGEVGALQEPPPPVWEGRAIATHKLRLVEFSAFMESRNREEAYQRHLFVHIGGPALSYSDPLLEAVDVRQIYDKFPEKKGGLKELYDKGPQNAFFLVKFWADLNSNFQDEAGAFYGVTSSYESNENMIITCSTKVCSFGKQVVEKVETEYARFENGRFVYRIHRSPMCEYMINFIHKLKHLPEK